(19) United States
(12) Patent Application Publication
(10) Pub. No.: US 2024/0189145 A1
(43) Pub. Date: Jun. 13, 2024

(54) TUNABLE DEVICE FOR TREATING EYE DISEASE

(71) Applicant: Avisi Technologies, Inc., Redwood City, CA (US)

(72) Inventors: Rui Jing Jiang, Lexington, MA (US); Brandon Kao, Placentia, CA (US); Georgia Griggs, Dover, DE (US); Jeffrey Lorne Emery, Emerald Hills, CA (US)

(21) Appl. No.: 18/553,085

(22) PCT Filed: Apr. 1, 2022
(86) PCT No.: PCT/US2022/023064

§ 371 Date: Sep. 28, 2023

Related U.S. Application Data
(60) Provisional application No. 63/169,587, filed on Apr. 1, 2021.

(51) Int. Cl. A61F 9/007

(52) U.S. Cl. CPC A61F 9/00781; A61F 2230/0017; A61F 2240/002; A61F 2250/0097

(57) ABSTRACT

Described herein are tunable treatment devices used to treat various diseases such as high intraocular pressure and glaucoma. A treatment device (1) includes a plate structure (200) that is modified based on a treatment application. The mechanical properties of the treatment device (1) may be tuned to achieve a desired bending stiffness, flow rate, and/or tensile elasticity. The mechanical properties may be tuned by changing an overall device shape, device thickness, core materials, and/or channel dimensions. For example, the treatment device (1) can be tuned to have a greater flow rate to treat patients that have higher intraocular pressures. In another example, a core thickness of the treatment device (1) may be increased, thereby increasing bending stiffness to improve surgical handling and reduce a likelihood of folding after implantation. Further, a tensile elasticity of the treatment device may be changed to reduce mechanical strain on surrounding tissue.

### BACKGROUND

Millions of individuals suffer from eye disease, specifically glaucoma. Most glaucoma patients have abnormally high intraocular pressure (IOP) due to the patient's inability to drain excessive aqueous humor from the anterior chamber of the eye through the trabecular meshwork. If not reduced with adequate treatment, high IOP will continuously damage the optic nerve as the disease progresses, leading to loss of vision or even total blindness. Current medications, surgeries, and implants have proven inadequate in lowering pressure within the eye or sustaining normal eye pressure over many years. Therefore, a need exists for new ways to alleviate IOP, thereby treating glaucoma.

### SUMMARY

Described herein are treatment devices, or simply devices, configured for treating ocular and other conditions. In one embodiment, an ocular condition is elevated intraocular pressure, and the devices herein are configured to lower the intraocular pressure. In another embodiment, a condition is hydrocephalus, and the devices herein are configured to lower pressure. The devices generally include a plate structure or core component comprising a first major surface coated with a first material and a second major surface coated with a second material.

In some embodiments, the treatment device disclosed herein is modified based on a treatment application. For example, the mechanical properties of the treatment device may be tuned to achieve a desired bending stiffness, flow rate, and/or tensile elasticity. The mechanical properties may be tuned by changing an overall device shape, device thickness, core materials, and/or channel dimensions. For example, the treatment device can be tuned to have a greater flow rate to treat patients that have higher intraocular pressures. In another example, a core thickness of the treatment device may be increased, thereby increasing bending stiffness to improve surgical handling and reduce a likelihood of folding after implantation. Further, a tensile elasticity of the treatment device may be changed to reduce mechanical strain on surrounding tissue. In another example, coatings may be modified based on a treatment application.

In some embodiments, some portions of a treatment device may be tuned in a first configuration while other portions of the treatment device are tuned in another configuration. The different tuning of portions of the treatment device enable certain portions of the device to be adapted to corresponding eye structure, thereby improving patient comfort and treatment outcomes. For example, tensile elasticity may be increased in certain locations that would otherwise impart a greater strain on a patient's eye tissue.

The plate structure or plate can have a thickness ranging from about 1 nanometer (nm) to about 10,000 nm, or from about 50 nm to about 800 nm.

The plate structure can include channels that assist with movement of ocular fluids which, in turn, reduces intraocular pressure.

Other embodiments include methods of reducing intraocular pressure. In one embodiment, a method includes securing a device, as described herein, to an eye thereby moving ocular fluids and reducing intraocular pressure.

In some embodiments, the plate structure is formed of a ceramic material. The ceramic material can be selected from aluminum oxide (alumina), silicon nitride, silica, hafnium oxide, titanium nitride, titanium, or combinations thereof.

In some embodiments, the first coating is a polymeric material. The polymeric material can be a parylene polymer. The parylene polymer can be parylene C, parylene D, parylene N, a derivative thereof or a combination thereof.

In other embodiments, the polymeric material includes rubber, synthetic rubber, silicone polymers, parylene, thermoplastics, thermosets, polyolefins, polyisobutylene, acrylic polymers, ethylene-co-vinylacetate, polybutylmethacrylate, vinyl halide polymers, polyvinyl ethers, polyvinylidene halides, polyacrylonitrile, polyvinyl ketones, polyvinyl aromatics, polyvinyl esters, acrylonitrile-styrene copolymers, ABS resins, ethylene-vinyl acetate copolymers, polyamides, alkyd resins, polycarbonates, polyoxymethylenes, polyimides, polyethers, epoxy resins, polyurethanes, rayon, cellulose, cellulose acetate, cellulose butyrate, cellulose acetate butyrate, cellophane, cellulose nitrate, cellulose propionate, cellulose ethers, carboxy methyl cellulose, polytetrafluororethylene, poly(ether-ether-ketone), poly lactides such as PLA, PLGA, PLLA, derivatives thereof, or combinations thereof.

In some embodiments, the second coating includes aluminum oxide and/or a parylene polymer.

In some embodiments, the second coating includes aluminum oxide in combination with rubber, synthetic rubber, silicone polymers, parylene, thermoplastics, thermosets, polyolefins, polyisobutylene, acrylic polymers, ethylene-co-vinylacetate, poly butylmethacrylate, vinyl halide polymers, polyvinyl ethers, polyvinylidene halides, polyacrylonitrile, polyvinyl ketones, polyvinyl aromatics, polyvinyl esters, acrylonitrile-styrene copolymers, ABS resins, ethylene-vinyl acetate copolymers, polyamides, alkyd resins, polycarbonates, polyoxymethylenes, polyimides, polyethers, epoxy resins, polyurethanes, rayon, cellulose, cellulose acetate, cellulose butyrate, cellulose acetate butyrate, cellophane, cellulose nitrate, cellulose propionate, cellulose ethers, carboxymethyl cellulose, polytetrafluororethylene, poly(ether-ether-ketone), poly lactides such as PLA, PLGA, PLLA, derivatives thereof, or combinations thereof.

The series of fluid channels can include a plurality of open-ended channels interconnected to form an intersecting network (or grid pattern) of fluid pathways. In some embodiments, the channels are microchannels.

In some embodiments, treating the high intraocular pressure is a treatment for glaucoma.

In light of the disclosure set forth herein, and without limiting the disclosure in any way, in a first aspect of the present disclosure, which may be combined with any other aspect, or portion thereof, described herein a device for lowering intraocular pressure includes a plate comprising a first surface opposite a second surface. The first surface includes interconnected fluid channels. The device also includes a first coating on the first surface and a second coating on the second surface. At least one of a thickness of the plate, dimensions of the plate, a thickness of the first coating, a thickness of the second coating, or dimensions of the fluid channels are selected to achieve a desired bending stiffness, a tensile elasticity, or fluid flow rate.

In a second aspect of the present disclosure, which may be combined with any other aspect, or portion thereof, described herein, the fluid channels form a hexagonal pattern with each channel having a height and first width to produce a desired fluid flow rate.

In a third aspect of the present disclosure, which may be combined with any other aspect, or portion thereof, described herein, a first portion of the plate has a first thickness and a second portion of the plate has a second thickness.

In a fourth aspect of the present disclosure, which may be combined with any other aspect, or portion thereof, described herein, the first thickness is greater than the second thickness.

In a fifth aspect of the present disclosure, which may be combined with any other aspect, or portion thereof, described herein, the first portion includes an extension portion and the second portion includes a main body portion.

In a sixth aspect of the present disclosure, which may be combined with any other aspect, or portion thereof, described herein, the at least one of the thickness of the plate, the dimensions of the plate, the thickness of the first coating, the thickness of the second coating, or the dimensions of the fluid channels as selected based on a treatment application or desired outcome.

In a seventh aspect of the present disclosure, which may be combined with any other aspect, or portion thereof, described herein, the plate is formed from a ceramic material selected from the group consisting of alumina, silicon nitride, silica, hafnium oxide, titanium nitride, and titanium carbide.

In an eighth aspect of the present disclosure, which may be combined with any other aspect, or portion thereof, described herein, the first coating has a thickness of about 0.1 μm to about 10 μm.

In a ninth aspect of the present disclosure, which may be combined with any other aspect, or portion thereof, described herein, the first coating is a parylene polymer including at least one of parylene C, parylene D, parylene N, a derivative thereof, or a combination thereof.

In a tenth aspect of the present disclosure, which may be combined with any other aspect, or portion thereof, described herein, the second coating is aluminum oxide or a parylene polymer.

In an eleventh aspect of the present disclosure, which may be combined with any other aspect, or portion thereof, described herein, at least one of the first coating or the second coating includes at least one of a biocompatible film, a porous coating, or a lubricious coating.

In a twelfth aspect of the present disclosure, which may be combined with any other aspect, or portion thereof, described herein, the biocompatible film includes polytetrafluoroethylene (PTFE) or enhanced PTFE.

In a thirteenth aspect of the present disclosure, which may be combined with any other aspect, or portion thereof, described herein, the series of fluid channels includes a plurality of open-ended channels that are interconnected to form an intersecting grid pattern of fluid pathways.

In a fourteenth aspect of the present disclosure, which may be combined with any other aspect, or portion thereof, described herein, at least one of the first surface or the second surface includes a marking that is visible under at least one of visible, ultraviolent, or infrared light for intraoperative or post-operative monitoring.

In a fifteenth aspect of the present disclosure, which may be combined with any other aspect, or portion thereof, described herein, the plate structure includes at least one notch along a perimeter, the notch being indicative as to whether the first surface or the second surface is visible to a surgeon.

In a sixteenth aspect of the present disclosure, which may be combined with any other aspect, or portion thereof, described herein, the plate structure includes at least one geometric feature for attachment to patient tissue.

In a seventeenth aspect of the present disclosure, which may be combined with any other aspect, or portion thereof, described herein, a method of fabricating a device for a treatment application for lowering tissue or organ fluid pressure includes receiving at least one of an input of mechanical properties or a treatment application for the device, determining dimensions of the device based on the input, preparing a mold, substrate, or wafer using photolithography and reactive ion etching to achieve the determined dimensions including a hexagonal pattern of fluid microchannels, depositing a layer of aluminum oxide to form the device, removing the mold, substrate, or wafer, coating the device with a parylene polymer, and cutting the device based on the determined dimensions.

In an eighteenth aspect of the present disclosure, which may be combined with any other aspect, or portion thereof, described herein, the treatment application includes at least one of: lowering intraocular pressure, glaucoma, hydrocephalus, plastic surgery drainage of hematoma, seroma, or serous fluids, cell growth, a delivery of cells, a delivery of nucleic acids, a delivery of nanoparticles, or a conduit for a delivery of drugs.

In a nineteenth aspect of the present disclosure, which may be combined with any other aspect, or portion thereof, described herein, the dimensions of the device include at least one of a thickness of the device, dimensions of the device, a thickness of the coating, dimensions of fluid microchannels channels, or dimensions of open cells.

In a twentieth aspect of the present disclosure, which may be combined with any other aspect, or portion thereof, described herein, the mechanical properties include at least one of a desired bending stiffness, a tensile elasticity, or a fluid flow rate.

In a twenty-first aspect of the present disclosure, which may be combined with any other aspect, or portion thereof, described herein, a first portion of the device is formed to have a first thickness and a second portion of the device is formed to have a second thickness.

In a twenty-second aspect of the present disclosure, which may be combined with any other aspect, or portion thereof, described herein, the first thickness is greater than the second thickness.

In a twenty-third aspect of the present disclosure, which may be combined with any other aspect, or portion thereof, described herein, the dimensions are additionally determined based on patient placement information.

In a twenty-fourth aspect of the present disclosure, which may be combined with any other aspect, or portion thereof, described herein, the layer of aluminum oxide is deposited at a specified thickness based on the determined dimensions.

In a twenty-fifth aspect any of the features, functionality and alternatives described in connection with any one or more of FIGS. 1 to 16 may be combined with any of the features, functionality and alternatives described in connection with any other of FIGS. 1 to 16.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE DRAWINGS

The present invention will become more fully understood from the detailed description and the accompanying drawings, wherein:

FIG. 1 is a perspective view of a treatment device according to one embodiment.

FIG. 2 is a close-up view of the device according to section A identified in FIG. 1.

FIG. 3 is a cross-sectional view of the device shown along line III-III in FIG. 2.

FIG. 4 is a perspective view of a device according to another embodiment.

FIG. 5A is a portion of a cross-sectional view of section A of the device shown in FIG. 4 according to one embodiment.

FIG. 5B is a portion of a cross-sectional view of section A of the device shown in FIG. 4 according to one embodiment.

FIG. 5C is a portion of a cross-sectional view of section A of the device shown in FIG. 4 according to one embodiment.

FIG. 6 is a diagram of a treatment device implanted in the anterior chamber and between conjunctival tissue and scleral tissue of a patient's eye, according to an example embodiment of the present disclosure.

FIG. 7 is a diagram of the treatment device for a first treatment application, according to an example embodiment of the present disclosure.

FIG. 8A shows the treatment device configured for another treatment application, according to an example embodiment of the present disclosure.

FIG. 8B shows the treatment device as a thin strip, according to an example embodiment of the present disclosure.

FIG. 9 is a diagram of an example procedure to fabricate the plate structure disclosed herein, according to an example embodiment of the present disclosure.

FIGS. 10 and 11 are diagrams of alternative embodiments of the treatment device of FIG. 1 that may be formed using the procedure of FIG. 9 to achieve a desired bleb formation, bending stiffness, tensile elasticity, fluid dispersion pattern, and/or fluid fluid flow rate, according to example embodiments of the present disclosure.

FIG. 12 is a diagram of a cross-section of the treatment device of FIGS. 10 and 11, according to an example embodiment of the present disclosure.

FIG. 13 is a diagram of an example pattern located on the treatment device of FIG. 12, according to an example embodiment of the present disclosure.

FIG. 14 is a diagram showing a surface texture of the treatment device formed from channels and open cells, according to an example embodiment of the present disclosure.

FIG. 15 is a diagram of a geometric feature of the treatment device for receiving a suture for attachment to a patient's tissue, according to an example embodiment of the present disclosure.

FIG. 16 is a diagram that shows various possible shapes and dimensions for the treatment device 1 that may be formed using the procedure of FIG. 9 to achieve a desired bending stiffness, tensile elasticity, and/or fluid flow rate, according to example embodiments of the present disclosure.

### DETAILED DESCRIPTION

The following description of the embodiment(s) is merely exemplary in nature and is in no way intended to limit the invention, its application, or uses.

Reference is made herein to lowering intraocular pressure. However, it should be appreciated that the disclosed devices and methods may low fluid pressure of other organs or tissue. For example, the disclosed devices and methods may be used to lower an accumulation of fluid in the brain.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below;” “up,” “down,” “top,” and “bottom” as well as derivatives thereof (e.g., “horizontally,” “downwardly,” “upwardly,” etc.) should be construed to refer to the orientation as then described or as shown in the drawing under discussion. These relative terms are for convenience of description only and do not require that the structure be constructed or operated in a particular orientation unless explicitly indicated as such.

Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features: the scope of the invention being defined by the claims appended hereto.

Unless otherwise specified, all percentages and amounts expressed herein and elsewhere in the specification should be understood to refer to percentages by weight. The amounts given are based on the weight of the material. According to the present application, the term “about” means+/−5% of the reference value. According to the present application, the term “substantially free” means less than about 0.1 wt. % based on the total of the referenced value.

A “subject” herein may be a human or a non-human animal, for example, but not by limitation, rodents such as mice, rats, hamsters, and guinea pigs: rabbits: dogs: cats: sheep; pigs: goats: cattle: horses: and non-human primates such as apes and monkeys, etc.

### Treatment Device Embodiment

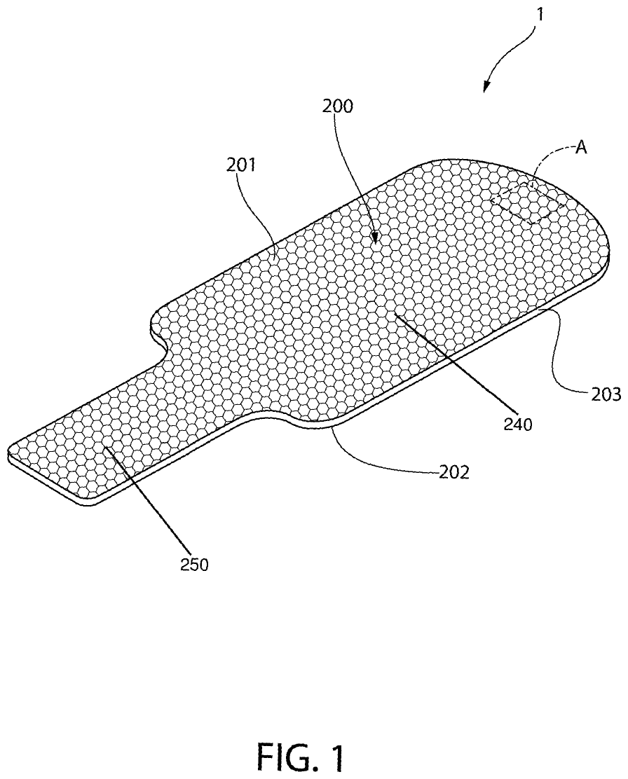

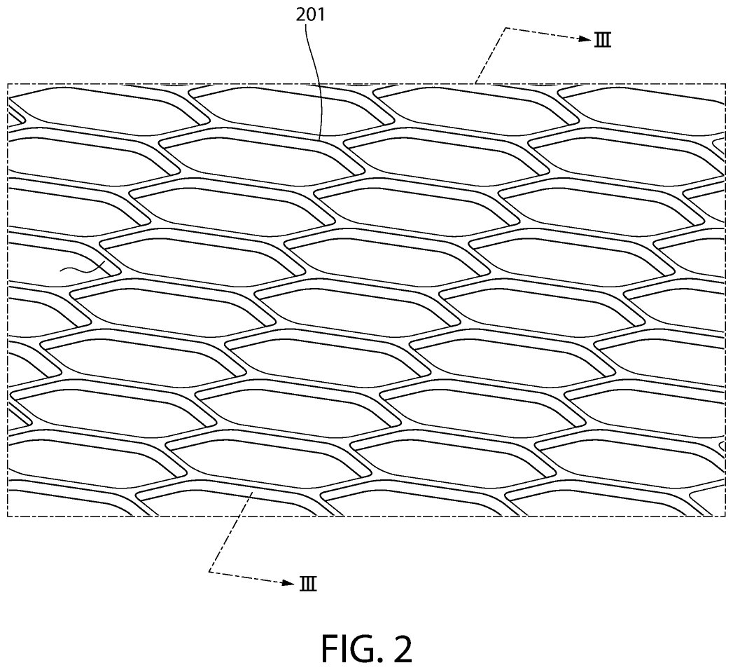

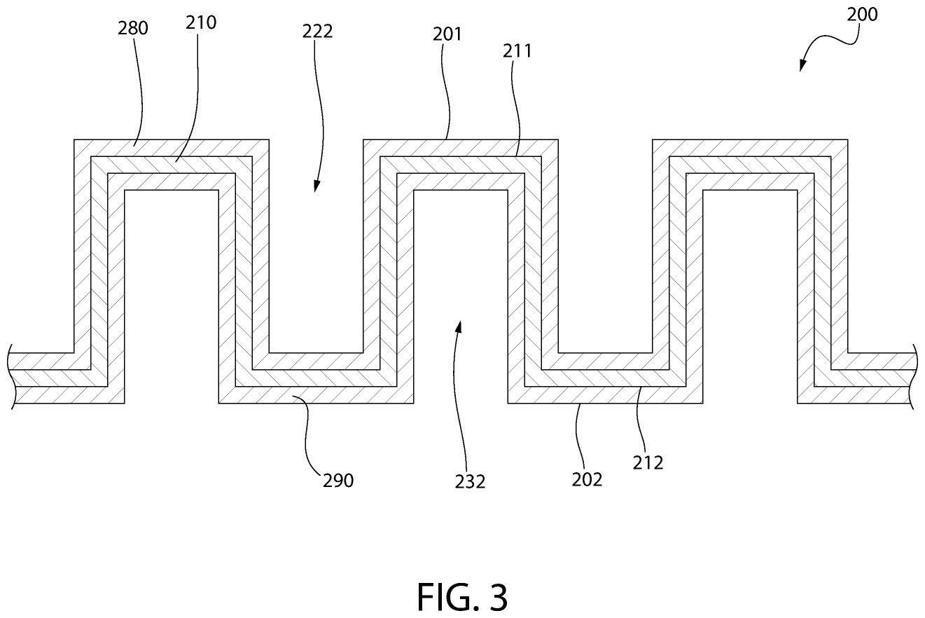

Referring to FIGS. 1-3, a treatment device 1 includes a plate structure 200, or simply plate, having a first major exposed surface 201 opposite a second major exposed surface 202 as well as side surface 203 extending there-between. The plate structure 200 can comprise an extension portion 250 and a main body portion 240.

The plate structure 200 can be formed of any material with appropriate characteristics for implantation and treatment. In some embodiments, the plate structure 200 can be formed of a metal, polymer, ceramic (e.g., aluminum oxide), other composite material, or a combination thereof. Metals can include, but are not limited to aluminum, titanium, zinc, platinum, tantalum, copper, nickel, rhodium, gold, silver, palladium, chromium, iron, indium, ruthenium, osmium, tin, iridium, or combinations, and alloys thereof. In some embodiments, alloys can include steel and nickel titanium such as Nitinol.

Polymers or polymer materials used to form plate structure 200 can include any of the polymers described herein.

Composites such as silicon composites can also be used. In one embodiment, a composite can include silicon nitride (Si₃N₄). The silicon nitride can have any known crystalline structure such as, but not limited to, trigonal α-Si₃N₄, hexagonal β-Si₃N₄, or cubic γ-Si₃N₄.

The plate structure 200, or plate, can have a thickness ranging from about 1 nm to about 1,000 nm, from about 1 nm to about 500 nm, from about 1 nm to about 400 nm, from about 100 nm to about 1,000 nm, from about 200 nm to about 1,000 nm, from about 300 nm to about 1,000 nm, from about 400 nm to about 1,000 nm, from about 1 nm to about 900 nm, from about 1 nm to about 800 nm, from about 1 nm to about 700 nm, from about 1 nm to about 600 nm, from about 300 nm to about 500 nm, from about 300 nm to about 600 nm, from about 400 nm to about 600 nm, from about 200 nm to about 600 nm, from about 200 nm to about 500 nm, or from about 50 nm to about 800 nm.

The plate structure 200 may comprise a multi-directional plate 210 comprising a first major surface 211 opposite a second major surface 212. The multi-directional plate 210 may form a plurality of topographical features (for example, a repeating honeycomb pattern) on each of the first major surface 211 and the second major surface 212. Each of the first and second topographies may independently comprise a plurality of channels 232 and/or a plurality of open-cells 222.

The plurality of channels 232 may be interconnected and can form a network of channels. The channels may be open or closed, allowing fluid to readily enter each channel of plurality of channels 232 and flow through it. The network may comprise intersecting channels in any suitable configuration to best help promote the flow of fluid across the plate structure 200 via the plurality of channels 232. In one embodiment, the channels 232 may be configured to form hexagonal patterns. Once treatment device 1, illustrated in FIG. 1, is implanted, fluid (e.g., aqueous humor) may be driven by a pressure gradient to flow through the channels and across the surface of plate structure 200.

In some embodiments, the channels 232 can include a ribbing pattern. The ribbing pattern and/or the geometry of the channels in the plate can be varied based on different severities of disease (e.g., mild, moderate, or severe glaucoma). In one embodiment, larger or smaller channels can be used to decrease intraocular pressure by different amounts. Changing intraocular pressure by a lower amount can decrease risk of hypotony (a condition that can exist if intraocular pressure is reduced too much) and increase efficacy at lowering pressure to a target level. In some embodiments, a device as described herein with smaller channels can decrease flow and decrease risk of hypotony. Likewise, larger channels can increase flow and allow the device to reduce intraocular pressure to a lower level.

The plate structure 200 may further comprise a first coating 280 applied to the first major surface 211 of the multi-directional plate 210. The first coating 280 may conform to the first topography of the first major surface 211 of the multi-directional plate 210. In other embodiments, the first coating 280 may form a topography that does not conform to the first topography of the first major surface 211 of the multi-directional plate 210.

The first coating 280 may have a thickness ranging from about 0.1 μm to about 10 μm or about 0.1 μm to about 2 μm—including all thickness and sub-ranges there-between. In one embodiment, the thickness is between about 0.4 μm (400 nm) and 0.6 μm (600 nm). In one embodiment, the thickness is about 0.4 μm (400 nm). In other embodiments, the thickness is between about 1 μm and about 5 μm, between about 1 μm and about 3 μm, between about 2 μm and about 5 μm, or between about 2 μm and about 4 μm. In one embodiment, the thickness is about 2 μm.

The plate structure 200 may further comprise a second coating 290 applied to the second major surface 212 of the multi-directional plate 210. The second coating 290 may conform to the plurality of surface features on the second major surface 212 of the multi-directional plate 210. In other embodiments, the second coating 290 may form a topography that does not conform to the second topography of the second major surface 212 of the multi-directional plate 210.

The second coating 290 may have a thickness ranging from about 0.1 μm to about 10 μm or about 0.1 μm to about 1 μm—including all thickness and sub-ranges there-between. In one embodiment, the thickness is between about 0.4 μm (400 nm) and 0.6 μm (600 nm). In one embodiment, the thickness is about 0.4 μm (400 nm). In other embodiments, the thickness is between about 1 μm and about 5 μm, between about 1 μm and about 3 μm, between about 2 μm and about 5 μm, or between about 2 μm and about 4 μm. In one embodiment, the thickness is about 2 μm.

In some embodiments, the plate structure 200 may comprise only the first coating 280—i.e., no second coating. In other embodiments, the plate structure 200 may comprise only the second coating 290—i.e., no first coating. In other embodiments, the plate structure 200 may comprise the first coating 280 and the second coating 290, whereby the first and second coatings overlap to fully encapsulate the multi-directional plate 210. In such embodiments, the side surface 203 of the plate structure 200 may comprise at least one of the first coating 280 and the second coating 290.

In some embodiments, the first and second coating, and any edge coating, can be thicker than the plate itself. In some embodiments, the coating thickness can be one, two or three orders of magnitude thicker than the plate structure. However, in other embodiments, the plate can be thicker than each coating or the additive thickness of the two coatings.

Coatings described herein can be applied by any suitable deposition method, such as but not limited to, physical vapor deposition, chemical vapor deposition, atomic layer deposition, spray coating, spin coating, self-assembly, dip coating, or brushing.

The first coating 280 may be applied to the first major surface 211 by any suitable deposition method. In a non-limiting example, the first coating 280 may be applied to the first major surface 211 by chemical vapor deposition, physical vapor deposition, or plasma-enhanced chemical vapor deposition. In another non-limiting example, the first coating 280 may be applied to the first major surface 211 by atomic layer deposition. In another non-limiting example, the first coating 280 may be applied to the first major surface 211 by spray coating. In another non-limiting example, the first coating 280 may be applied to the first major surface 211 by dip coating. In another non-limiting example, the first coating 280 may be applied to the first major surface 211 by brushing.

The second coating 290 may be applied to the second major surface 212 by any suitable deposition method. In a non-limiting example, the second coating 290 may be applied to the second major surface 212 by chemical vapor deposition, physical vapor deposition, or plasma-enhanced chemical vapor deposition. In another non-limiting example, the second coating 290 may be applied to the second major surface 212 by atomic layer deposition. In another non-limiting example, the second coating 290 may be applied to the second major surface 212 by spray coating. In another non-limiting example, the second coating 290 may be applied to the second major surface 212 by dip coating. In another non-limiting example, the second coating 290 may be applied to the second major surface 212 by brushing.

The first coating 280 may be the same as the second coating 290. The first coating 280 and the second coating 290 may be different. The first coating 280 may be hydrophilic. The first coating 280 may be hydrophobic. The first coating 280 may be lipophilic. The first coating 280 may be lipophobic. The second coating 290 may be hydrophilic. The second coating 290 may be hydrophobic. The second coating 290 may be lipophilic. The second coating 290 may be lipophobic. Each of the first and second coatings 280, 290 may independently be continuous. Each of the first and second coatings 280, 290 may independently be discontinuous. In some embodiments, the first and second coatings 280, 290 may both be hydrophobic. In some embodiments, the first and second coatings 280, 290 may both be hydrophilic. In some embodiments, the first and second coatings 280, 290 may both be lipophilic or lipophobic.

The first coating 280 may be organic. The first coating 280 may be inorganic. The second coating 290 may be organic. The second coating 290 may be inorganic.

In some embodiments, the first coating 280 is hydrophilic and the second coating 290 is hydrophobic. In some embodiments, the first coating 280 is hydrophilic and the second coating 290 is hydrophilic. Having at least one of the first and/or second coating 280, 290 be hydrophobic may help prevent the treatment device 1 from inadvertently sticking to tissue during implantation.

In some embodiments, a purpose of a first and/or second coating is to increase the toughness of the device. Also, a first and/or second coating can increase biocompatibility of the device and/or decrease scarring by decreasing tissue and/or fibroblast adhesion. In some embodiments, the coatings described herein are hydrophobic and decrease tissue adhesion. In some embodiments, tissue adhesion can be reduced by greater than about 10%, greater than about 60%, greater than about 70%, greater than about 80%, greater than about 90%, greater than about 95%, greater than about 96%, greater than about 97%, greater than about 98%, or greater than about 99% when compared to an uncoated plate.

In a non-limiting embodiment, the first and/or second coating may comprise a polymer, such as a parylene polymer (poly(para-xylylene)) or a derivative thereof. In other embodiments, the first and/or second coating can include aluminum oxide, a biocompatible film, a porous coating, or a lubricious coating. In one embodiment, the parylene polymer is a chlorine modified poly(para-xylylene), or a fluorine modified poly(para-xylylene). In one embodiment, the parylene polymer can be parylene C, parylene D, parylene N, a derivative thereof or a combination thereof. In other embodiments, the first and/or second coating can include aluminum oxide.

In other embodiments, other polymer(s) can be used in addition to, in combination with, or instead of a parylene polymer and/or aluminum oxide. In some embodiments, other polymeric materials can include, but are not limited to rubber, synthetic rubber, silicone polymers, thermoplastics, thermosets, polyolefins, polyisobutylene, acrylic polymers, ethylene-co-vinylacetate, poly butylmethacrylate, vinyl halide polymers (for example, polyvinyl chloride), polyvinyl ethers (for example, polyvinyl methyl ether), polyvinylidene halides, polyacrylonitrile, polyvinyl ketones, polyvinyl aromatics, polyvinyl esters, acrylonitrile-styrene copolymers, ABS resins, ethylene-vinyl acetate copolymers, polyamides (for example, Nylon 66 and polycaprolactam), alkyd resins, polycarbonates, polyoxymethylenes, polyimides, polyethers, epoxy resins, polyurethanes, rayon, cellulose, cellulose acetate, cellulose butyrate, cellulose acetate butyrate, cellophane, cellulose nitrate, cellulose propionate, cellulose ethers, carboxymethyl cellulose, polytetrafluororethylene (for example, Teflon), poly(ether-ether-ketone), poly lactides such as PLA, PLGA, PLLA, derivatives thereof, or combinations thereof.

The resulting the treatment device 1 may comprise the first plurality of channels 222 present on the first exposed major surface 201 of the plate structure 200, wherein the first plurality of channels 222 are hydrophilic due to the presence of the first coating 280. The resulting treatment device 1 may comprise the second plurality of channels 232 present on the second exposed major surface 202 of the plate structure 200, wherein the second plurality of channels 232 are hydrophilic due to the presence of the second coating 290. As discussed, the hydrophilic channels may promote fluid flow through the channels after the treatment device 1 has been implanted into a subject's eye.

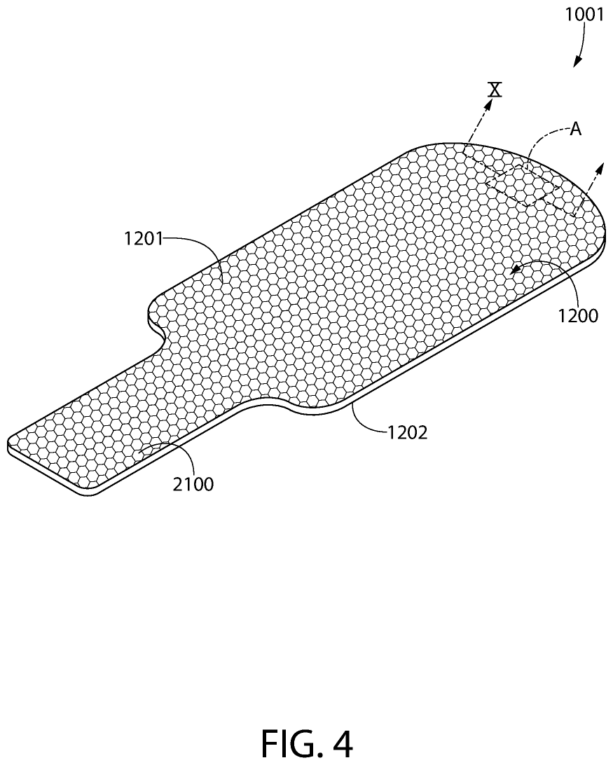

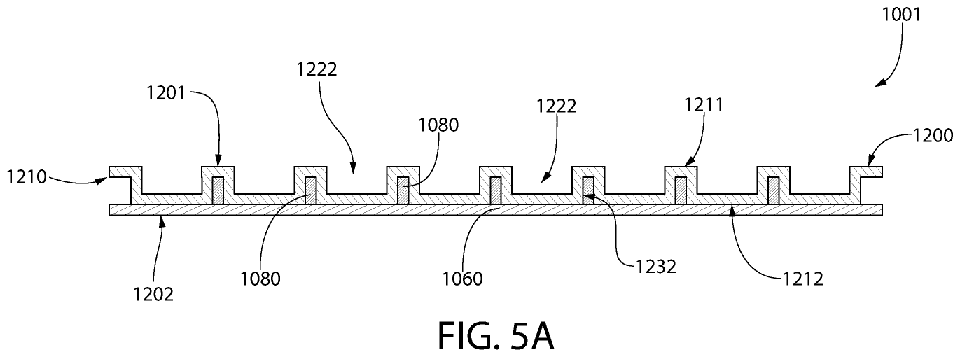

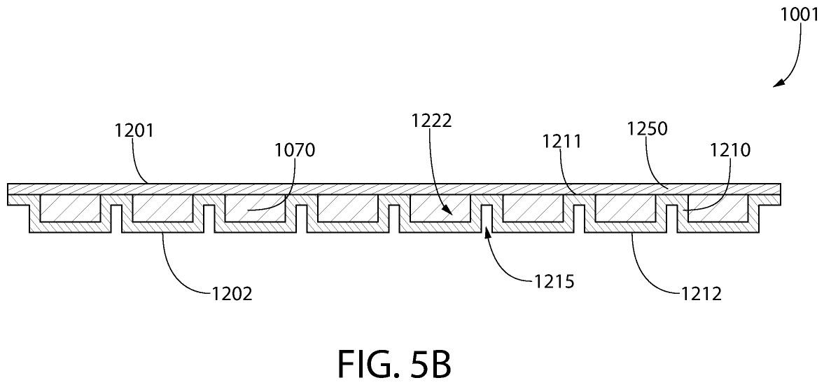

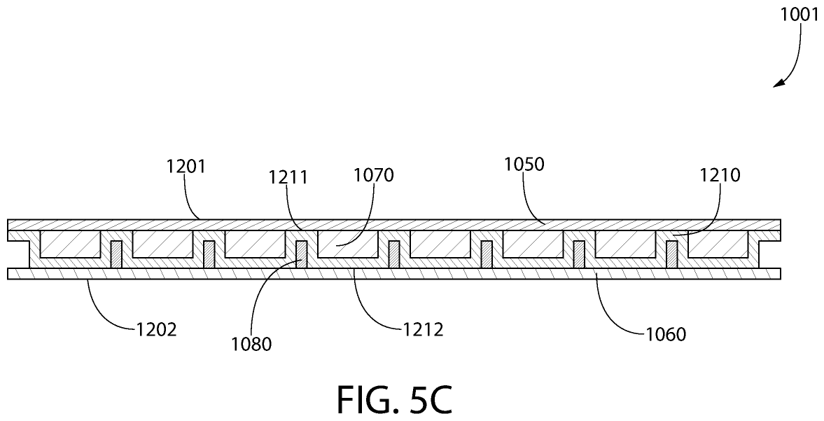

Referring to FIGS. 4, 5A, 5B, and 5C, generally, a treatment device 1001 is illustrated in accordance with another embodiment. The treatment device 1001 is similar to the treatment device 1 except as described herein below. The description of the treatment device 1 above generally applies to the treatment device 1001 described below except with regard to the differences specifically noted below. A similar numbering scheme will be used for the treatment device 1001 as with the treatment device 1 except that a “1000” series of numbering will be used.

The treatment device 1001 comprises a plate structure 1200 having a first exposed major surface 1201 that is opposite a second exposed major surface 1202. The plate structure 1200 may comprise a multi-directional plate 1210 comprising a first major surface 1211 opposite a second major surface 1212. The multi-directional plate 1210 may form a plurality of topographical features (for example, a repeating honeycomb pattern) on each of the first major surface 1211 and the second major surface 1212. Each of the first and second topographies may independently comprise a plurality of channels 1232 and/or a plurality of open-cells 1222.

Referring now to FIG. 5B, the plate structure 1200 may comprise a first delivery component 1070 present in the open voids created by the first topography formed by the first exposed surface 1211 of the multi-directional plate 1210. Specifically, the first delivery component 1070 may be present in the open voids created by the open-cells 1222 of first topography formed by the first major surface 1211 of the multi-directional plate 1210.

The first delivery component 1070 may comprise one or more active agents such as, but not limited to therapeutic and/or pharmacological components. The first delivery component 1070 may occupy some, all, or substantially all of the free volume present in the open-cells 1222 formed by the first topography.

In other embodiments, active agents can include any compound or drug having a therapeutic effect in a subject. Non limiting active agents include anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, steroids, proteasome inhibitors, antibiotics, anti-inflammatoires, anti-sense nucleotides, transforming nucleic acids, messenger ribonucleic acids, IOP lowering drugs, prostaglandins, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, cells, stem cells, liposomes, anti-metabolites such as mitomycin-C, combinations thereof, prodrugs thereof, pharmaceutical salts thereof, derivatives thereof, and the like.

The treatment device 1001 may further comprise a first coating 1050 applied to a first major surface 1211 of the multi-directional plate 1210. The first coating 1050 may cover both a first major surface 1211 of the multi-directional plate 1210 as well as a first delivery component 1070 that is present in the open-cells 1222 formed into the first major surface 1211 of the multi-directional plate 1210. The first coating 1050 may be in the form of a continuous film. The first coating 1050 may be flat. In other embodiments, the first coating 1050 may be conformal to the underlying pattern formed by the multi-directional plate 1210 and the first delivery component 1070.

Referring now to FIG. 5A, the plate structure 1200 may comprise a second delivery component 1080 present in the open voids created by the second topography formed by the second exposed surface 1212 of the multi-directional plate 1210. Specifically, the second delivery component 1080 may be present in the open voids created by the open-channels 1232 of the second topography formed by the second major surface 1212 of the multi-directional plate 1210. The second delivery component 1080 may be the same or different from the first delivery component 1070.

The second delivery component 1080 may comprise one or more therapeutic and/or pharmacological components—including but not limited to anti-inflammatory agents, steroids, antibiotics, analgesics. The second delivery component 1080 may occupy some, all, or substantially all of the free volume present in the channels 1232 formed by the first topography.

The treatment device 1001 may further comprise a second coating 1060 applied to a second major surface 1212 of the multi-directional plate 1210. The second coating 1060 may cover both the second major surface 1212 of the multi-directional plate 1210 as well as the second delivery component 1080 that is present in the open-channels 1232 formed into the second major surface 1212 of the multi-directional plate 1210. The second coating 1060 may be in the form of a continuous film. The second coating 1060 may be flat. In other embodiments, the second coating 1060 may be conformal to the underlying pattern formed by the multi-directional plate 1210 and the second delivery component 1080.

The second coating 1060 may be the same or different than the first coating 1050. For each of the first and the second coatings 1050, 1060, the resulting film may be formed from a slow-release material that dissolves slowly after exposure to aqueous humor or other biological fluids, thereby releasing the first delivery component 1070 from the channels 1232 of the treatment device 1001 after it has been implanted into a subject.

Referring now to FIG. 5C, in other embodiments, the treatment device 1001 may comprise both the first and the second delivery components 1070, 1080, as well as the first and the second coatings 1050, 1060 to encapsulate the first and second delivery components 1070, 1080.

In other embodiments, the plate structure 1200 may comprise at least one of the first coating 1050 and/or the second coating 1060 without the presence of the first and/or second delivery components 1070, 1080. In such embodiments, the first coating 1050 and/or the second coating 1060 may form a film that covers the open cells 1222 and/or the open channels 1232 created by the multi-directional plate.

The presence of the films resulting from the first and/or the second coating 1050, 1060 may enhance the overall strength of the resulting treatment device. Specifically, layered structure(s) of the films formed by the first and second coatings 1050, 1060, which are bonded to the first and second major surfaces 1211, 1212 of the multi-directional plate 1210, provide added mechanical integrity to the resulting treatment device.

Beyond achieving the baseline flexibility to conform to curvature of the eye, the addition of the first and/or second coatings 1050, 1060 may provide a mechanism that allows the overall treatment device to match the elastic modulus of surrounding tissues (e.g., conjunctival and scleral tissues) to maximize biocompatibility or biointegration. Findings in brain implant research confirm that the flexibility of implants in soft tissue improves compliance of the implant with microscale movements of surrounding tissue and reduces tissue displacement and trauma as well as facilitates implantation of the treatment device.

### Treatment Device Tuning Embodiment

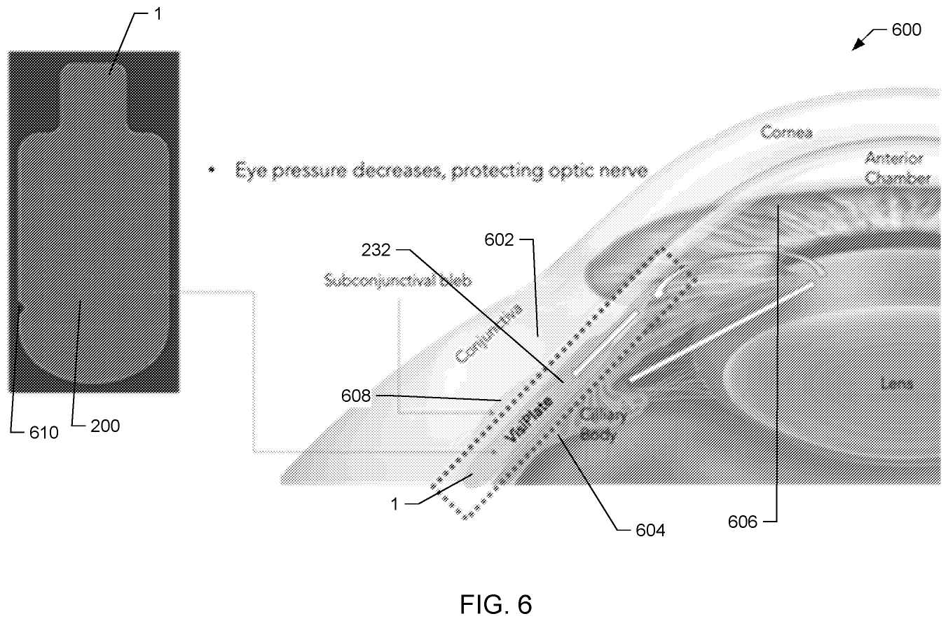

FIG. 6 is a diagram of the treatment device 1 implanted between conjunctival tissue 602 and scleral tissue 604 of a patient's eye 600, according to an example embodiment of the present disclosure. The treatment device 1 is a biocompatible ocular implant that includes a thin, flexible plate to facilitate safe, comfortable, and effective treatment. The treatment device 1 includes a plate structure 200 having a plurality of channels 232. The example channels 232 are configured to facilitate the draining of accumulated aqueous in the anterior chamber 606 of the eye 600 to a pocket (bleb) 608 that is located between the conjunctival tissue 602 and scleral tissue 604. This enables intraocular pressure from the accumulation of the aqueous in the anterior chamber 606 to be reduced. The removed aqueous in the pocket 608 is gradually reabsorbed by surrounding tissue, which enables further accumulating aqueous to be removed from the anterior chamber 606. This continuous draining of aqueous (e.g., glaucoma drainage) lowers pressure within the eye 600 and protects the optic nerve. The redundant channels 232 of the plate structure 200 prevent single-end clogging by scar tissue. Further, the thin profile of the plate structure 200 hinders tissue erosion.

FIG. 6 also shows the plate structure 200 including a notch 610 along a perimeter. While the notch 610 is shown on a lower left section of the plate structure 200, it should be appreciated that the notch 610 may be located at any location of the perimeter. Further, while one notch 610 is shown, the plate structure 200 may include two or more notches. The notch 610 is configured to facilitate proper installation and placement of the plate structure 200 within a patient's eye. The notch 610 may be indicative as to whether the channels 232 of the plate structure 200 are aligned upwards or downwards. The notch 610 accordingly provides confirmation to a clinician that the plate structure 200 is properly orientated.

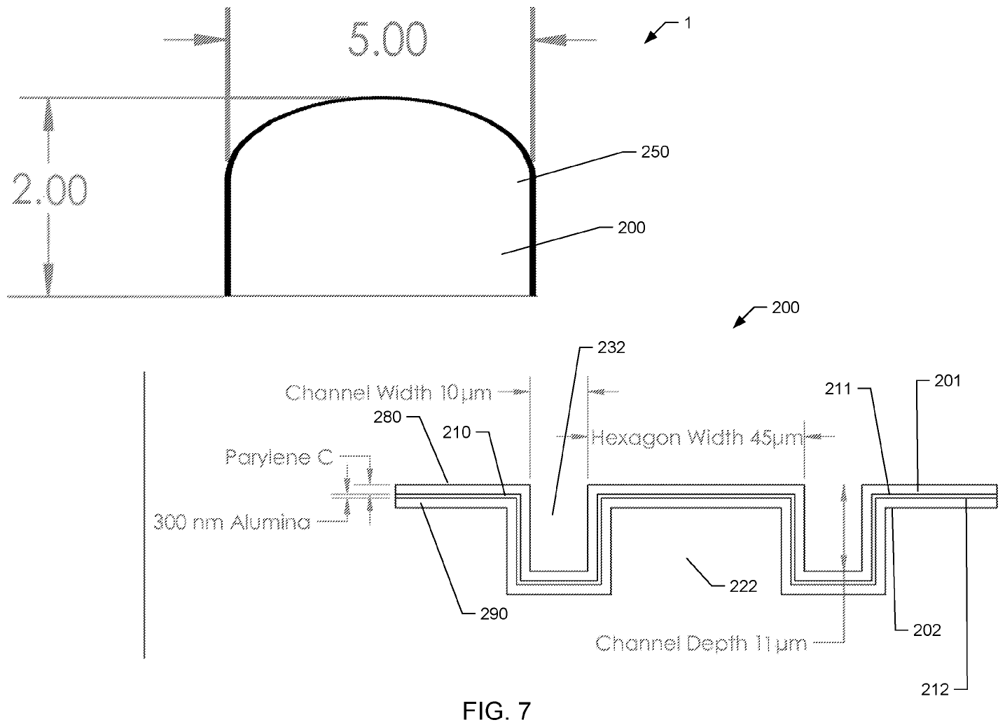

FIG. 7 is a diagram of the treatment device 1 for a first treatment application, according to an example embodiment of the present disclosure. The treatment device 1 includes a plate structure 200 having interconnected channels 232. In some embodiments, the channels 232 are provided in a grid pattern. The plate structure 200 may comprise a first major exposed surface 201 that is opposite a second major exposed surface 202. The plate structure 200 also includes a multi-directional plate 210 comprising a first major surface 211 that is opposite a second major surface 212. The multi-directional plate 210 may form a plurality of topographical features (for example, a repeating honeycomb pattern) on each of first major surface 211 and second major surface 212. Each of the first and second topographies may independently comprise plurality of channels 232 and/or plurality of open-cells 222.

The plate structure 200 may further comprise a first coating 280 applied to the first major surface 211 of the multi-directional plate 210. The first coating 280 may conform to the first topography of the first major surface 211 of the multi-directional plate 210. In other embodiments, the first coating 280 may form a topography that does not conform to the first topography of the first major surface 211 of the multi-directional plate 210. The plate structure 200 may further comprise a second coating 290 that is applied to the second major surface 212 of the multi-directional plate 210. The second coating 290 may conform to the plurality of surface features on the second major surface 212 of the multi-directional plate 210. In other embodiments, the second coating 290 may form a topography that does not conform to the second topography of the second major surface 212 of the multi-directional plate 210.

The multi-directional plate 210 may include a freestanding aluminum oxide (alumina Al₂O₃) plate with a corrugated structure to form the channels 232 and/or the plurality of open-cells 222. The coating 280 and/or 290 may include parylene-C. The coating 280 and/or 290 may also include at least one of parylene C, parylene D, parylene N, a derivative thereof, or a combination thereof. In some embodiments, at least one of the coating 280 or 290 includes aluminum oxide, a biocompatible film, a porous coating, or a lubricious coating. The biocompatible film may include polytetrafluoroethylene (PTFE) or enhanced PTFE, for example.

The corrugated pattern includes the network of channels 232, connected in-plane, to form a hexagonal honeycomb. The plate structure 200 has certain mechanical properties including tensile elasticity, bending stiffness, and flow rate. These mechanical properties may be tuned by varying dimensions and/or shapes of the channels, a thickness of the multi-directional plate 210, and/or a thickness of the coating 280 and/or 290. The mechanical properties may also be changed by changing a shape of the plate structure 200. The mechanical properties, may also be changed by changing the geometry of the plate structure 200. The Young's Modulus of the corrugated multi-directional plate 210 is based on Equation (1) below. In this equation, a is about 0.234 and B is about 0.062. W_{rib} corresponds to a width of a rib that forms the channels 232 and the hexagonal pattern. Dₕₑₓ corresponds to a diameter of each hexagon or other shape of the open-cells 222. h corresponds to a rib height or is a height of the channel and t corresponds to rib or plate thickness, such as thickness of an alumina core.

$$\frac{{E}^{*}}{{E}_{s}} = α \frac{I}{{tW}_{rib}^{3}} + β \frac{{h}^{4} {W}_{rib}^{4}}{{t}^{2} {D}_{hex}^{5} ( {W}_{rib} + 2 h )} \qquad ( 1 )$$

$$I = \frac{{tW}_{rib}^{3}}{12} + \frac{{t}^{3} h}{6} + \frac{{thW}_{rib}^{2}}{2} \qquad ( 2 )$$

A bending stiffness of the corrugated multi-directional plate 210 is based on a diameter and width of the hexagonal pattern, which is expressed in Equation (3) below:

$${BSEF}_{hex} \approx {( \frac{{D}_{hex}}{{W}_{rib}} + 1 )}^{2} \qquad ( 3 )$$

The flow rate of the corrugated multi-directional plate 210 is based on the Hagen Poiseuille equation modified for rectangular channels, and is shown in Equation (4) below where w is the width of the rib, h is the height of the channel, L is the length of the channel, Δp is the pressure differential in the system, μ is viscosity of the fluid, and λₙ is the eigenvalue term in the series.

$$Q = \frac{{wh}^{3} Δ p}{12 μ L} [ 1 - 6 ( \frac{h}{w} ) {\sum }_{n = 0}^{∞} {λ}_{n}^{- 5} tanh ( \frac{{λ}_{n} w}{h} ) ] \qquad ( 4 )$$

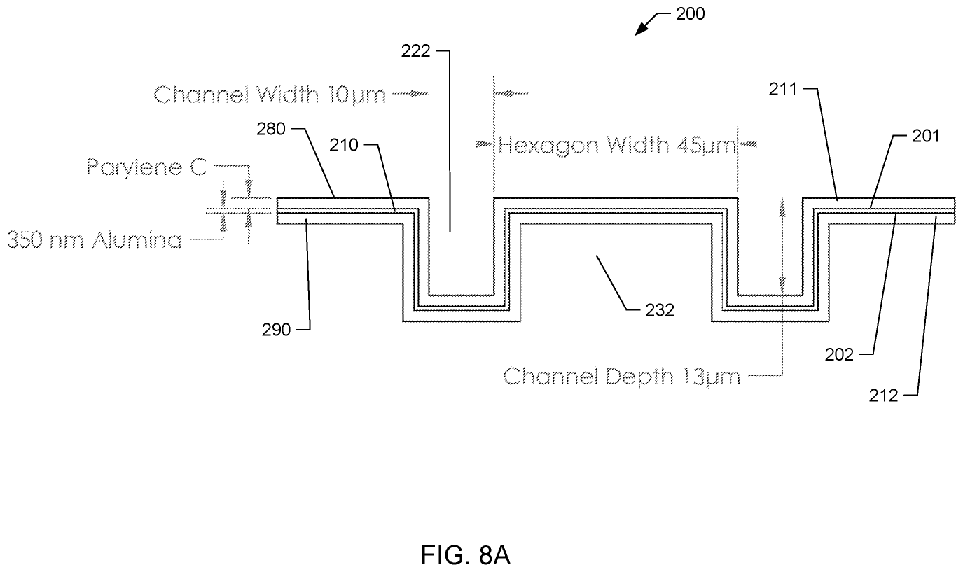

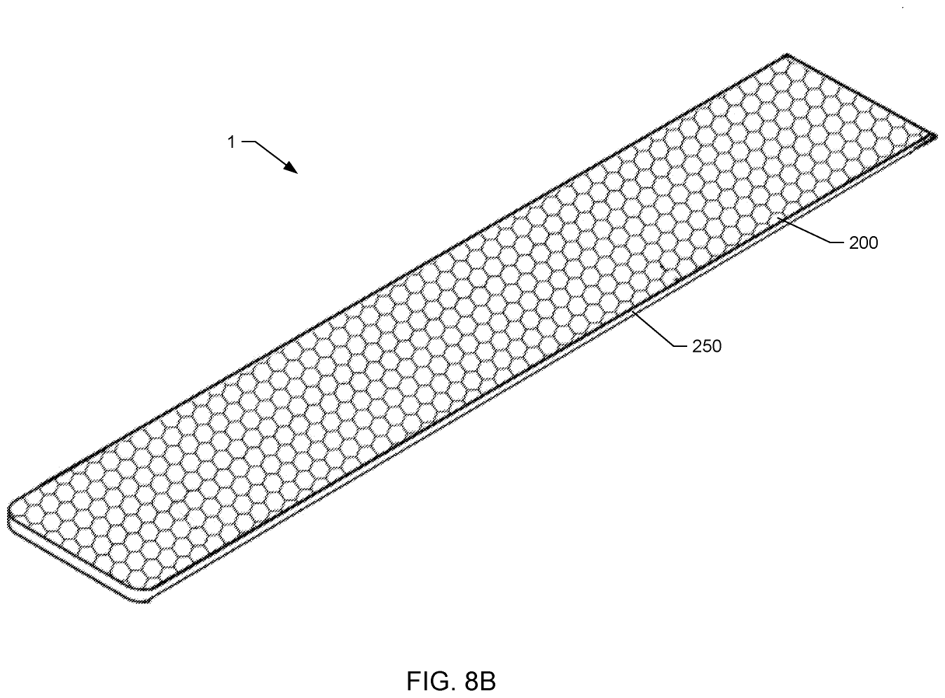

In the example of FIG. 7A, the plate structure 200 has channel dimensions of 10×14 μm, a neck width of 5 mm, and an internal alumina core thickness of 300 nm. This results in a flow rate of 2.91 L/min when implanted in an anterior chamber of a patient's eye. This plate structure 200 has a Young's Modulus of 22.10 gigapascal (GPa) and bending stiffness of 9.30*10⁻⁹ Nm\hat{ }2, which may be suited for providing a treatment for patients with a target intraocular pressure around 10 mmHg. If the channel x-axis dimensions are adjusted by only 2 μm to be 10×16 μm and the alumina core is changed to 350 nm (as shown in FIG. 8A), the flow rate is 3.60 μL/min, the Young's Modulus is 24.96 GPa, and the bending stiffness is 1.48*10\hat{ }−8 Nm\hat{ }2. It should be appreciated that small changes can have a significant effect on device performance, but also enables the plate structure 200 to be micro-tuned to meet surgical and/or functional performance requirements.

If a patient's glaucoma necessitates a greater reduction in intraocular pressure, the channel dimensions of the plate structure 200 may be increased to slightly facilitate shunting additional aqueous. In another embodiment, changing a thickness of the multi-directional plate 210 on the order or nanometers results in a stiffer plate structure 200, which may provide improved surgical handling and reduce the likelihood of folding.

In yet another embodiment, changing channel dimensions or thickness affects the Young's Modulus, which can be fine-tuned in select areas of the plate structure 200 to reduce the mechanical strain on surrounding tissue. For example, the thickness may be increased in the extension portion 250 and reduced in the main body portion 240. This may reduce the strain placed on the pocket 608 without affecting a flow rate. In other examples, the extension portion 250 may be thinner than the main body portion 240. Moreover, in some embodiments, different portions of the main body portion 240 may have different thicknesses.

In other embodiments, the plate structure 200 may include a thin rectangular strip (e.g., 100 μm×4 mm) for placement in other anatomical zones, as shown in FIG. 8B. For example, a thin rectangular strip may be used with other existing glaucoma technologies to keep a drainage space open. The thin rectangular strip may be dispensed from a syringe or needle for ab-interno insertion into the eye, to places such as the subconjunctival space or the superciliary space. The thin rectangular strip may also be used as a shunting mechanism for other diseases such as hydrocephalus, to enable draining fluid from ventricles in the brain to a surface of the head to lower pressure. The thin rectangular strip may also be used for surgical drain following plastic surgery (e.g., to drain hematoma, seroma, or serous fluids). It should be appreciated that the dimensions and mechanical characteristics of the plate structure 200 can be customized according to Equations (1) to (4) above based on use case. The use of the plate structure 200 as a surgical drain minimizes scarring in the patient and improves aesthetics compared to larger tubes.

The example plate structure 200 may also be configured as a retinal pigment epithelium (RPE) scaffold and/or a scaffold for cell growth in the posterior chamber, retina, macula, and/or optic nerve, for treatment of eye disease. Different channel patterning may be provided based on cell type and/or outcomes desired. Further, the plate structure 200 may be coated with chemotherapy drugs, nucleic acids, and/or nanoparticles for treating or identifying ocular tumors. Moreover, for open angle glaucoma patients, the dimensions of the plate structure 200 may be modified based on the opening of the drainage angle of the patient. The plate structure 200 may further be configured for treatment of hydrocephalus and/or to provide a plastic surgery drainage of hematoma, seroma, or serous fluids. The plate structure 200 may also provide for a delivery or administration of nanoparticles or drugs to the anterior chamber.

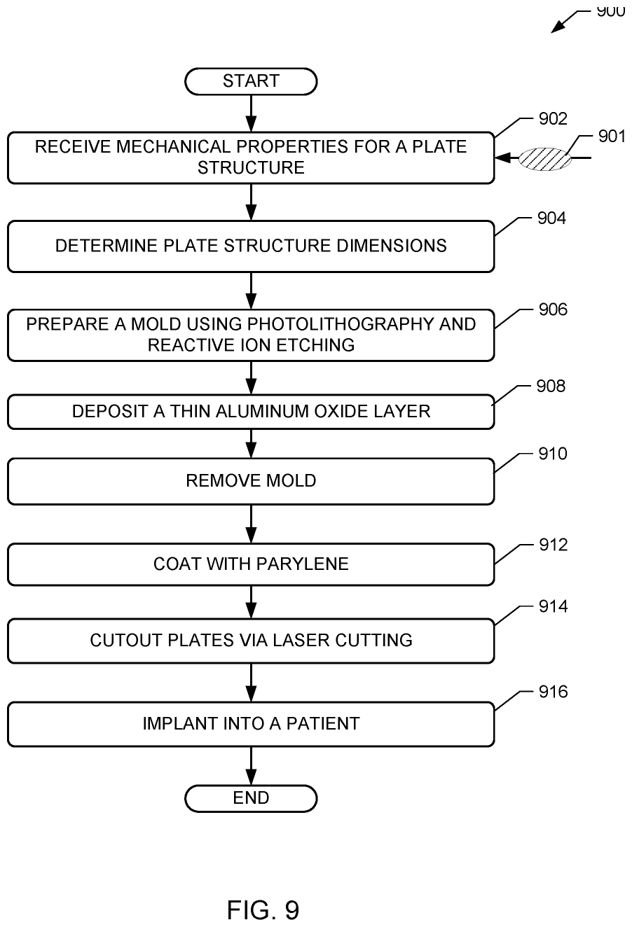

FIG. 9 is a diagram of an example procedure 900 to fabricate the plate structure 200 disclosed herein, according to an example embodiment of the present disclosure. Although the procedure 900 is described with reference to the flow diagram illustrated in FIG. 9, it should be appreciated that many other methods of performing the steps associated with the procedure 900 may be used. For example, the order of many of the blocks may be changed, certain blocks may be combined with other blocks, and many of the blocks described may be optional. In any embodiment, the number of blocks may be changed.

The example procedure 900 begins when desired mechanical properties 901 are entered into a computer system (block 901). The mechanical properties 901 may specify a desired flow rate of fluid to be achieved by a plate structure 200 of the treatment device 1 described above. The mechanical properties 901 may also specify a desired geometry for delivery of the materials described (drugs, cells, etc.). The mechanical properties 901 may also specify a bending stiffness or tensile elasticity for one or more areas. In some instances, the mechanical properties 901 may specify a treatment application.

The example procedure 900 determines dimensions for the plate structure 200 based on the mechanical properties 902 (block 904). The dimensions may include an overall width, an overall length, an overall shape, an extension portion width or length, a channel width and/or height, open cell geometry, plate thickness, coating thickness, etc. The procedure 900 next prepares a wafer, substrate, or mold using photolithography (block 906). This includes preparing a wafer, coating with photoresist, prebaking, aligning a mask, and exposing to light, and developing to remove portions of the photoresist. The removed photoresist portions define locations and widths of channels and geometries of other structures of the plate structure 200. As such, the resist mask is created based on the determined dimensions. Next, deep reactive ion etching is used to etch the wafer or mold in areas exposed by the removed photoresist. The depth of etching determines channel height and plate overall thickness. Accordingly, depth is controlled based on the determined dimensions. After etching, the remaining photoresist is removed.

The example process 900 continues by depositing a thin layer (e.g. of aluminum oxide) on the mold or wafer (block 908). Backside etching releases the thin membrane (block 910). In some embodiments, the aluminum oxide is deposited at a desired thickness to achieve a desired bending stiffness and/or tensile elasticity. In some embodiments, the thin membrane is coated with a polymer (e.g. parylene C) using, for example, conformal chemical vapor deposition, physical vapor deposition, or plasma-enhanced chemical vapor deposition (block 912). In other embodiments, the thin membrane is coated with at least one of a biocompatible film, a porous coating, or a lubricious coating. The thin membrane is then laser cut into desired dimensions to form the plate structure 200 (block 914). In other embodiments, the thin membrane is laser cut before coating. It should be appreciated that other types of cutting may be used instead of laser cutting such as stamping, punching, or milling. In addition to being cut into desired dimensions, one or more geometric features may be cut. The features may include the notch 610 and/or a pore, slot, hole, barb, etc. for connection to patient tissue.

The example plate structure 200 may then be implanted into a patient for treatment (block 916). The example procedure 900 then ends. As discussed above, the example plate structure 200 is tuned specifically to the medical treatment of the patient, thereby improving patient comfort and a treatment outcome.

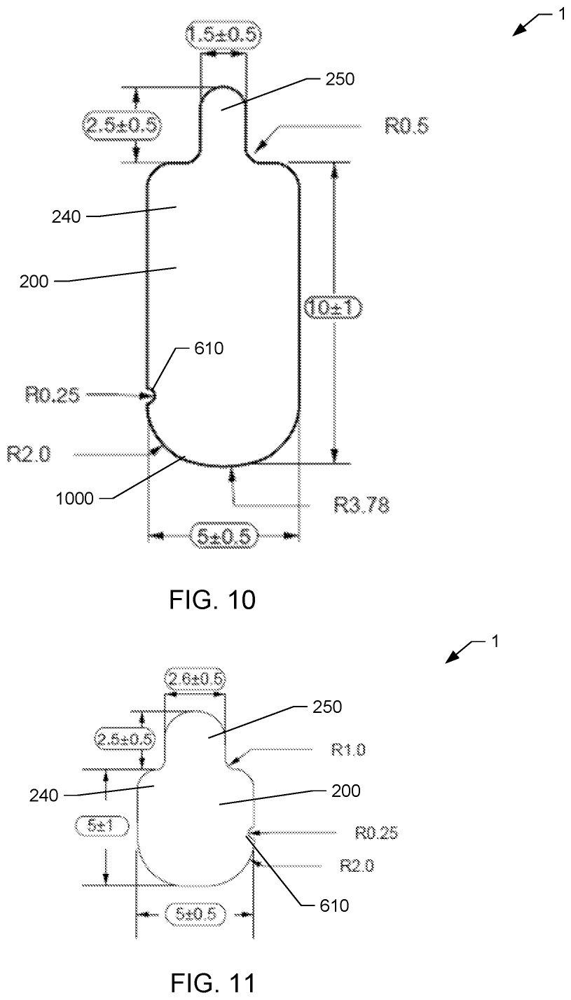

FIGS. 10 and 11 are diagrams of alternative embodiments of the treatment device 1 that may be formed using the procedure 900 of FIG. 9 to achieve a desired bending stiffness, tensile elasticity, and/or flow rate, according to example embodiments of the present disclosure. The treatment device 1 of FIG. 10 includes a plate structure 200 having an extension portion 250 with a width of 1.5±0.5 mm and a length of 2.5±0.5 mm. The plate structure 200 has a main body portion 240 with a width of 5±0.5 mm and a length of 10±1 mm. Further, a first end 1000 of the plate structure has a radius of curvature of 3.78 mm. FIG. 10 also shows the plate structure 200 including a notch 610. The dimensions of the plate structure 200 are selected, for example, to reduce bending stiffness or tensile elasticity. Additionally, the relative long length of the plate structure 200 may provide for moving fluids, such as aqueous humor, relatively longer distances.

The treatment device 1 of FIG. 11 has a relatively wider extension portion 250 of the plate structure 200. Further, the main body portion 240 is relatively shorter. The dimensions of the plate structure 200 of FIG. 11 are selected, for example, to increase bending stiffness or tensile elasticity.

FIG. 16 is a diagram that shows various possible shapes and dimensions for the treatment device 1 that may be formed using the procedure 900 of FIG. 9 to achieve a desired bending stiffness, tensile elasticity, and/or fluid flow rate, according to example embodiments of the present disclosure. The dimensions of the treatment device 1 are selected, for example, to posteriorize collection of fluid, reduce the area of potential foreign body reaction, and/or diffuse a fluid over a wider surface area, respectively. It should be appreciated that the illustrated designs are merely exemplary of the virtually endless number of designs and possible dimensions for the treatment device 1.

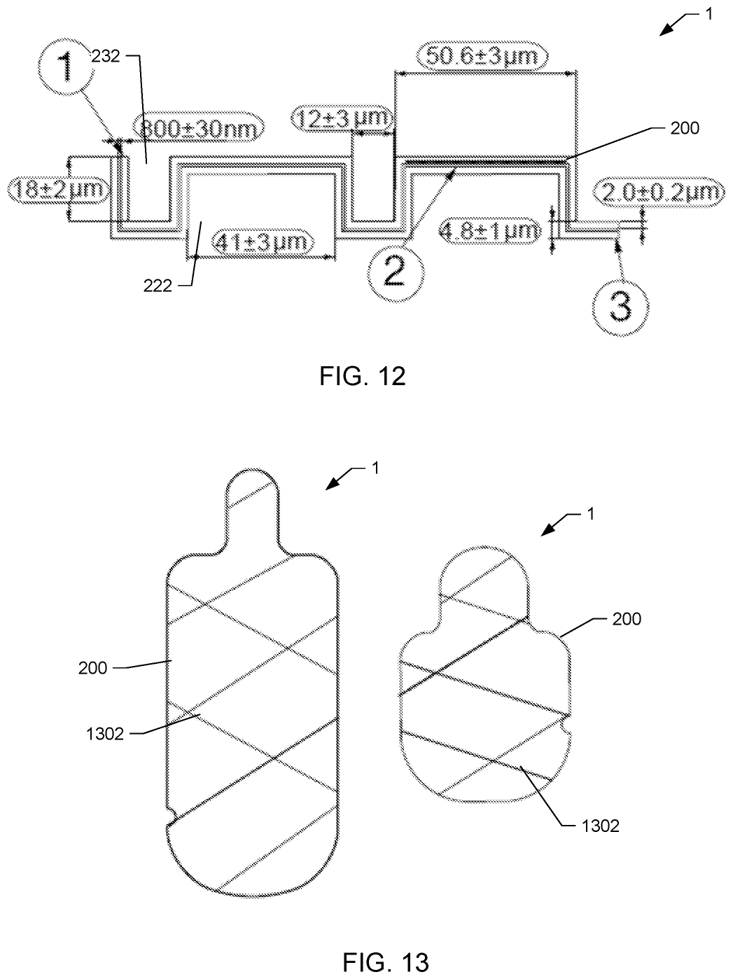

FIG. 12 is a diagram of a cross-section of the plate structure 200 of FIGS. 10 and 11, according to an example embodiment of the present disclosure. The cross-section may be taken along any length of the plate structure 200 shown in FIGS. 10 and 11. As shown the plate structure 200 includes an alumina core, referenced as number ‘1’. The alumina core may be derived from RO00002 alumina precursor and have a thickness of 800±30 nm. The alumina core is coated on at least one side by a parylene-C coating, referenced as number ‘3’. The coating has a thickness of 2±0.2 μm. The plate structure 200 includes open cells 222 having a width of 41±3 μm and channels 232 having a width of 12±3 μm. In some embodiments, the width of the channels 232 and cells 222 may be varied based on desired tensile flow rates, bending stiffness, and/or tensile elasticities.

FIG. 12 also shows that the plate structure 200 may include a surgical ink or other material marking to enhance visibility, referenced as number (2). The marking may be placed on a portion (or all) of the coating of the plate structure 200. In some embodiments, the marking may be placed on both sides of the plate structure 200.

FIG. 13 is a diagram of an example pattern of surgical ink 1302 on the plate structure 200 of FIG. 12, according to an example embodiment of the present disclosure. The surgical ink 1302 may include one or more lines. A clinician may view the surgical lines for post-operative monitoring through eye tissue. For example, a clinician may shine an ultraviolet light on a patient's eye post-surgery to view the surgical ink 1302 for determining whether the plate structure 200 has shifted. In some embodiments, the surgical ink 1302 may include a doping agent to ceramic and/or a coating. The surgical ink 1302 may be configured to be visible via visible light, ultraviolet light, and/or infrared light or intraoperative or post-operative monitoring.

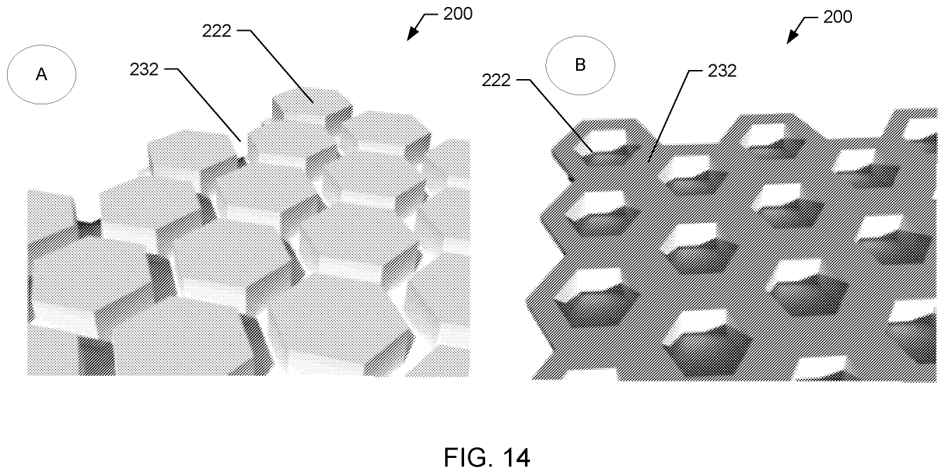

As described above, the plate structure 200 includes channels 232 that provide for the flow of aqueous humor from the anterior chamber of a patient's eye. FIG. 14 is a diagram showing a surface texture of the plate structure 200 formed from the channels 232 and the open cells 222, according to an example embodiment of the present disclosure. Diagram A shows a first or top side of the plate structure 200. Diagram B shows a second or underside of the plate structure 200. As shown, the plate structure 200 includes interconnected channels 232 that are arranged in a grid pattern. Open cells 222 are defined by the channels 232. In this embodiment, the open cells 222 form a honeycomb pattern of hexagons. In other examples, the pattern may include circles, rectangles, triangles, etc. The open cells 222 provide structural rigidity and can serve as depot for active therapies while the channels 232 enable aqueous humor or other liquids to flow through.

Dimensions of the open cells 222 relative to the channels 232 may be modified to change flow rates, bending stiffness, and/or tensile elasticity of the plate structure 200. For example, the channels 232 may be widened to increase a flow rate. However, wider channels may also reduce bending stiffness and/or increase tensile elasticity.

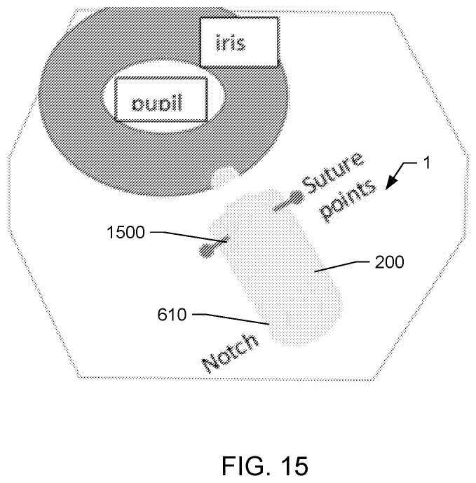

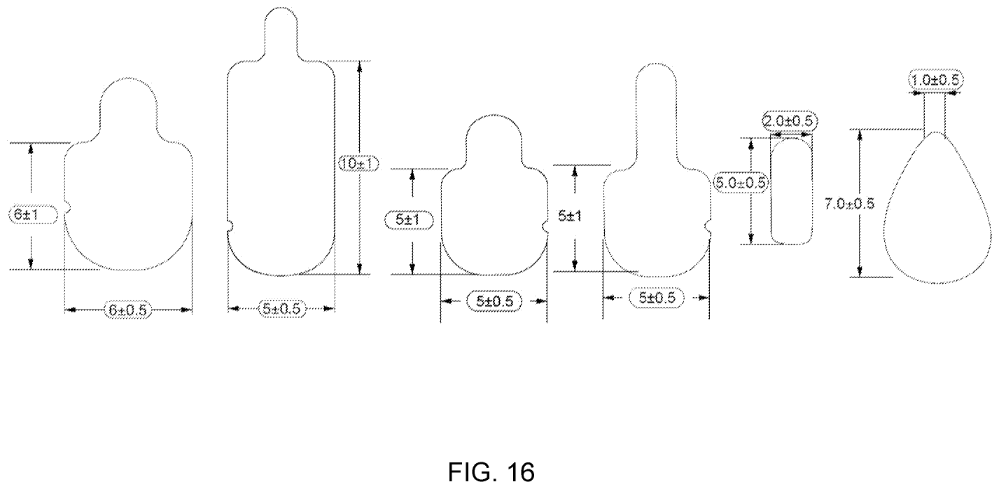

In some embodiments, the plate structure 200 may include one or more geometric features to provide attachment to a patient's tissue. FIG. 15 is a diagram of a geometric feature 1500 of the plate structure 200 for receiving a suture for attachment to a patient's tissue, according to an example embodiment of the present disclosure. The geometric feature 1500 may include an aperture or hole with a reinforced perimeter for receiving a suture. Alternatively, the geometric feature may include a well, a pore, a slot, a barb, or a groove that is configured to connect directly to a patient's eye tissue. In some embodiments, the geometric feature 1500 is configured to promote cellular ingrowth to prevent migration of the plate structure 200. Such geometric features 1500 may reduce the need for sutures.

### CONCLUSION

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a.” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific example embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Example embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1: A device for lowering intraocular pressure, the device comprising:
a plate comprising a first surface opposite a second surface, wherein the first surface includes interconnected fluid channels;
a first coating on the first surface; and
a second coating on the second surface,
wherein at least one of the first surface or the second surface includes a marking that is visible under at least one of visible, ultraviolent, or infrared light for intraoperative or post-operative monitoring.

2: The device according to claim 1, wherein the fluid channels form a hexagonal pattern with each channel having a height and first width to produce a desired fluid flow rate.

3: The device according to claim 1, wherein a first portion of the plate has a first thickness and a second portion of the plate has a second thickness.

4: The device according to claim 3, wherein the first thickness is greater than the second thickness.

5: The device according to claim 3, wherein the first portion includes an extension portion and the second portion includes a main body portion.

6: The device according to claim 1, wherein the interconnected fluid channels have dimensions between 10×14 and 12×18 micrometers (“μm”) and the plate includes a neck width between 2 and 5 millimeters (“mm”).

7: The device according to claim 1, wherein the plate is formed from a ceramic material selected from the group consisting of alumina, silicon nitride, silica, hafnium oxide, titanium nitride, and titanium carbide.

8: The device according to claim 1, wherein the first coating has a thickness of about 0.1 μm to about 10 μm.

9: The device according to claim 1, wherein the first coating is a parylene polymer including at least one of parylene C, parylene D, parylene N, a derivative thereof, or a combination thereof.

10: The device according to claim 1, wherein the second coating is aluminum oxide or a parylene polymer.

11: The device according to claim 1, wherein at least one of the first coating or the second coating includes at least one of a biocompatible film, a porous coating, or a lubricious coating.

12: The device according to claim 11, wherein the biocompatible film includes polytetrafluoroethylene (PTFE) or enhanced PTFE.

13: The device of claim 1, wherein the series of fluid channels includes a plurality of open-ended channels that are interconnected to form an intersecting grid pattern of fluid pathways.

14: The device of claim 1, wherein the plate has a core thickness between 300 and 800 nanometers.

15: The device of claim 1, wherein the plate structure includes at least one notch along a perimeter, the notch being indicative as to whether the first surface or the second surface is visible to a surgeon.

16: The device of claim 1, wherein the plate structure includes at least one geometric feature for attachment to patient tissue.

17: A method of fabricating a device for a treatment application for lowering tissue or organ fluid pressure, the method comprising:
receiving at least one of an input of mechanical properties or a treatment application for the device;
determining dimensions of the device based on the input;
preparing a mold, substrate, or wafer using photolithography and reactive ion etching to achieve the determined dimensions including a hexagonal pattern of fluid microchannels;
depositing a layer of aluminum oxide to form the device;
removing the mold, substrate, or wafer;
coating the device with a parylene polymer; and
cutting the device based on the determined dimensions.

18: The method according to claim 17, wherein the treatment application includes at least one of: lowering intraocular pressure, glaucoma, hydrocephalus, plastic surgery drainage of hematoma, seroma, or serous fluids, cell growth, a delivery of cells, a delivery of nucleic acids, a delivery of nanoparticles, or a conduit for a delivery of drugs.

19: The method of claim 17, wherein the dimensions of the device include at least one of a thickness of the device, dimensions of the device, a thickness of the coating, dimensions of fluid microchannels channels, or dimensions of open cells.

20: The method of claim 17, wherein the mechanical properties include at least one of a desired bending stiffness, a tensile elasticity, or a fluid flow rate.

21: The method of claim 17, wherein a first portion of the device is formed to have a first thickness and a second portion of the device is formed to have a second thickness.

22: The method according to claim 21, wherein the first thickness is greater than the second thickness.

23: The method of claim 17, wherein the dimensions are additionally determined based on patient placement information.

24: The method of claim 17, wherein the layer of aluminum oxide is deposited at a specified thickness based on the determined dimensions.
